(19) United States
(12) Patent Application Publication
(10) Pub. No.: US 2003/0165443 A1
(43) Pub. Date: Sep. 4, 2003

(54) Composition containing a dibenzoylmethane derivative and an alpha-alkylstyrene dimer uv stabilising method

(76) Inventor: Serge Forestier, Claye Souilly (FR)

Correspondence Address: BURNS DOANE SWECKER & MATHIS L L P, POST OFFICE BOX 1404, ALEXANDRIA, VA, 22313-1404, US

(21) Appl. No.: 10/129,483

(22) PCT Filed: Aug. 23, 2001
(86) PCT No.: PCT/FR01/02655

(30) Foreign Application Priority Data
Sep. 5, 2000 (FR) 00/11304
Dec. 21, 2000 (FR) 00/16791

(51) Int. Cl. A61K 7/42

(52) U.S. Cl. 424/59

(57) ABSTRACT

The invention relates to a cosmetic or dermatological composition, for topical use, in particular for the photoprotection of skin and hair, characterized in that it comprises at least, in a cosmetically acceptable support:

(a) from 0.1 to 20% by weight of a UV screening agent of the dibenzoylmethane-based type and

(b) from 0.1 to 20% by weight of one specific α-alkylstyrene-based dimer.

The invention relates to a novel process for improving the stability of at least one dibenzoylmethane derivative towards UV radiation, which consists in combining with the said dibenzoylmethane derivative an effective amount of at least one specific α-alkylstyrene-based dimer.

The invention relates to a cosmetic or dermatological composition, for topical use, in particular for the photoprotection of skin and hair, characterized in that it comprises at least, in a cosmetically acceptable support:

(a) from 0.1 to 20% by weight of a UV screening agent of the dibenzoylmethane-based type and

(b) from 0.1 to 20% by weight of one specific α-alkylstyrene-based dimer.

The invention relates to a novel process for improving the stability of at least one dibenzoylmethane derivative towards UV radiation, which consists in combining with the said dibenzoylmethane derivative an effective amount of at least one specific α-alkylstyrene-based dimer.

It is known that light radiation with wavelengths of between 280 nm and 400 nm permit tanning of the human epidermis and that light rays with wavelengths more particularly between 280 and 320 nm, known as UV-B rays, cause skin burns and erythema which can harm the development of a natural tan. For these reasons, and also for aesthetic reasons, there is a constant demand for means for controlling this natural tanning in order thus to control the colour of the skin; this UV-B radiation should thus be screened out.

It is also known that UV-A rays, with wavelengths between 320 and 400 nm, which cause browning of the skin, are liable to induce adverse changes therein, in particular in the case of sensitive skin or skin which is continually exposed to solar radiation. UV-A rays in particular cause a loss of elasticity of the skin and the appearance of wrinkles, leading to premature ageing of the skin. They promote triggering of the erythemal reaction or amplify this reaction in certain individuals and may even be the cause of phototoxic or photoallergic reactions. Thus, for aesthetic and cosmetic reasons such as the conservation of the skin's natural elasticity, for example, an increasingly large number of people wish to control the effect of UV-A rays on their skin. It is thus desirable also to screen out UV-A radiation.

In this regard, one particularly advantageous family of UV-A screening agents currently consists of dibenzoylmethane derivatives, and in particular 4-(tert-butyl)-4′-methoxydibenzoylmethane, which have high intrinsic absorbing power. These dibenzoylmethane derivatives, which are now products that are well known per se as screening agents that are active in the UV-A range, are described in particular in French patent applications FR-A-2 326 405 and FR-A-2 440 933, and also in European patent application EP-A-0 114 607; 4-(tert-butyl)-4′-methoxydibenzoylmethane is moreover currently sold under the trade name “Parsol 1789” by the company Hoffmann LaRoche.

However, it is found that dibenzoylmethane derivatives are products that are relatively sensitive to ultraviolet radiation (especially UV-A), that is to say, more specifically, that they have an unfortunate tendency to degrade more or less quickly under the action of this radiation. Thus, this substantial lack of photochemical stability of dibenzoylmethane derivatives towards the ultraviolet radiation to which they are by nature intended to be subjected does not make it possible to ensure continual protection during prolonged exposure to the sun, such that the user has to make repeated applications at regular and close time intervals in order to obtain effective protection of the skin against UV rays.

The photo-stabilization of dibenzoylmethane derivatives towards UV radiation constitutes, at the present time, a problem which has still not been solved entirely satisfactorily.

Now, the Applicant has just discovered, surprisingly, that by combining with the dibenzoylmethane derivatives mentioned above an effective amount of at least one specific α-alkylstyrene-based dimer which will be defined later, it is possible to obtain a substantial and noteworthy improvement in the photochemical stability (or photostability) of these same dibenzoylmethane derivatives.

Thus, in accordance with one of the subjects of the present invention, a novel cosmetic or dermatological composition is now proposed, for topical use, in particular for the photoprotection of skin and hair, characterized in that it comprises at least, in a cosmetically acceptable support:

(a) from 0.1 to 20% by weight of a UV screening agent of the dibenzoylmethane-based type and

(b) from 0.1 to 20% by weight of one specific α-alkylstyrene-based dimer.

Another subject of the invention is a novel process for stabilizing dibenzoylmethane derivatives against UV radiation (wavelengths between 280 nm and 400 nm approximately), and in particular solar radiation, characterized in that it consists in combining with the said dibenzoylmethane derivatives an effective amount of at least one α-alkylstyrene-based dimer of formula (I) defined below.

In accordance with the invention, the expression “effective amount of α-alkylstyrene-based dimer” means an amount which is sufficient to obtain an appreciable and significant improvement in the photostability of the dibenzoylmethane derivative(s) of the photo-protective cosmetic composition. This minimum amount of photo-stabilizer to be used, which may vary according to the nature of the cosmetically acceptable support selected for the composition, may be determined without any difficulty by means of a conventional test for measuring photostability, such as the one given in the examples below.

Finally, a subject of the present invention is also the use of an α-alkylstyrene-based dimer of formula (I) defined below, in the preparation of a cosmetic or dermatological composition comprising at least one dibenzoylmethane derivative, with the aim of improving the stability towards UV rays of the said dibenzoylmethane derivative contained.

Other characteristics, aspects and advantages of the present invention will become apparent on reading the detailed description which follows.

The α-alkylstyrene-based dimer compounds in accordance with the invention correspond to formula (I) below:

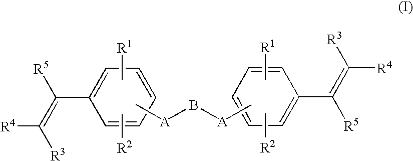

in which

R¹ and R², which may be identical or different, denote hydrogen, OH, NH₂, a linear or branched C₁-C₁₂ alkyl radical; a linear or branched C₁-C₁₂ alkoxy radical; a linear or branched C₁-C₁₂ monoalkylamino radical; a linear or branched C₁-C₁₂ dialkylamino radical or a water-solubilizing substituent chosen from a carboxylate group, a sulphonate group and an ammonium residue;

R³ and R⁴, which may identical or different, denote a group COOR⁶ COR⁶ CONR⁶R⁷ or CN;

R⁵ denotes a linear or branched C₁-C₁₂ alkyl radical;

R⁶ and R⁷, which may be identical or different, denote hydrogen, a linear or branched C₁-C₁₂ alkyl radical; a linear or branched C₂-C₁₀ alkenyl radical; a branched or unbranched C₃-C₁₀ cycloalkyl radical; a branched or unbranched C₃-C₁₀ cycloalkenyl; a C₆-C₁₈ aryl or a C₄-C₇ heteroaryl; the said cycloalkyl, cycloalkeny and aryl groups possibly comprising one or more substituents chosen from halogens, a cyano group, a nitro group, an amino group, a C₁-C₄ monoalkylamino radical, a C₁-C₄ dialkylamino radical, a hydroxyl group, a C₁-C₄ alkyl radical and a C₁-C₄ alkoxy radical; the said cycloalkyl and cycloalkenyl groups possibly comprising one or more hetero atoms (for example O, N or S);

A denotes O, S or a group NR⁸;

R⁸ denotes hydrogen or a linear or branched C₁-C₁₂ alkyl radical;

B denotes a linear or branched C₁-C₁₂ alkylene radical which may comprise one or more substituents chosen from hydroxyl, OC₁-C₆-acyl, NH₂, NH-C₁-C₆-alkyl, NH-C₁-C₆-acyl, CN, COOH and COO-C₁-C₆-acyl; a branched or unbranched C₄-C₁₂ cycloalkylene radical; a C₈-C₂₂ aralkylene radical; a C₉-C₂₁ monooxoaralkylene radical; or a group [X]ₙ—Y—;

at least two of the radicals R¹, R² and R⁸ may together form a 5- or 6-membered ring with the benzene nucleus to which they are attached;

X denotes a group —CH₂—CH₂—Z—, —CH₂CH₂CH₂Z—, —CH(CH₃)—CH₂—Z—, —CH₂CH₂—CH₂—CH₂—Z— or CH₂—CH(CH₂CH₃)—Z—;

Y denotes a group —CH₂—CH₂—, —CH₂CH₂CH₂—, —CH(CH₃)—CH₂—, —CH₂—CH₂—CH₂—CH₂— or —CH₂—CH(CH₂CH₃)—;

Z denotes O or S;

n ranges from 1 to 150.

Examples of C₁-C₁₂ alkyl radicals which may be mentioned for the radicals R¹, R², R⁶, R⁸ and B are: methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, (1,2,2-trimethylpropyl), 1-ethyl-1-methylpropyl, 1-ethyl-2-methylpropyl, n-heptyl, n-octyl, n-nonyl, n-decyl, n-undecyl or n-dodecyl.

Examples of C₂-C₁₀ alkenyl groups which may be mentioned for the radicals R⁶ and R⁷ are: ethenyl, n-propenyl, 1-methylethenyl, n-butenyl, 1-methylpropenyl, 2-methylpropenyl, 1,1-dimethylethenyl, n-pentenyl, 1-methylbutenyl, 2-methylbutenyl, 3-methylbutenyl, 2,2-dimethylpropenyl, 1-ethylpropenyl, n-hexenyl, 1,1-dimethylpropenyl, 1,2-dimethylpropenyl, 1-methylpentenyl, 2-methylpentenyl, 3-methylpentenyl, 4-methylpentenyl, 1,1-dimethylbutenyl; 1,2-dimethylbutenyl, 1,3-dimethylbutenyl, 2,2-dimethylbutenyl, 2,3-dimethylbutenyl, 3,3-dimethylbutenyl, 1-ethylbutenyl, 2-ethylbutenyl, 1,1,2-trimethyl-propenyl, 1,2,2-trimethylpropenyl, 1-ethyl-1-methyl-propenyl, 1-ethyl-2-methylpropenyl, n-heptenyl, n-octenyl, n-nonenyl or n-decenyl.

Examples of C₃-C₁₀ cycloalkyl radicals which may be mentioned for the radicals R⁶ and R⁷ are: cyclopropyl, cyclobutyl, cyclopentyl, cyclohexyl, cycloheptyl, 1-methylcyclopropyl, 1-ethylcyclopropyl, 1-propylcyclopropyl, 1-butylcyclopropyl, 1-pentylcyclopropyl, 1-methyl-1-butylcyclopropyl, 1,2-dimethylcyclopropyl, 1-methyl-2-ethylcyclopropyl, cyclooctyl, cyclononyl or cyclodecyl.

As C₃-C₁₀ cycloalkenyl radicals containing one or more double bonds, for the radicals R⁶ and R⁷, mention may be made of: cyclobutenyl, cyclopentenyl, cyclopentadienyl, cyclohexenyl, 1,3-cyclohexadienyl, 1,4-cyclohexadienyl, cycloheptenyl, cycloheptatrienyl, cyclooctenyl, 1,5-cyclooctadienyl, cyclooctatetraenyl, cyclononenyl or cyclodecenyl.

As C₁-C₁₂ alkoxy radicals for the radicals R¹ and R², mention may be made of: methoxy, n-propoxy, 1-methylpropoxy, n-pentoxy, 3-methylbutoxy, 2,2-dimethylpropoxy, 1-methyl-1-ethylpropoxy, octoxy, ethoxy, n-propoxy, n-butoxy, 2-methylpropoxy, 1,1-dimethylpropoxy, hexoxy, heptoxy or 2-ethylhexoxy.

The aryl groups for the radicals R⁶ and R⁷ are more particularly phenyl, methoxyphenyl or naphthyl.

The water-solubilizing groups denoted by the radicals R¹ and R² are, for example, carboxylate or sulphonate groups and more particularly salts thereof with physiologically acceptable cations, for instance the alkali metal salts or trialkylammonium salts such as the tris(hydroxyalkyl)ammonium or 2-methyl-1-propanol-2-ammonium salts. Mention may also be made of ammonium groups, for instance alkylammoniums and forms thereof salified with physiologically acceptable anions.

As C₄-C₁₂ cycloalkyl for the radical B, mention may be made, for example, of:

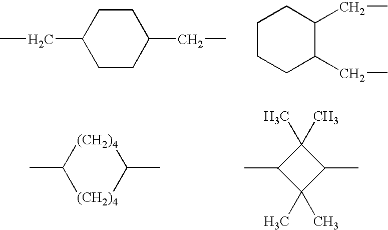

As C₈-C₂₂ aralkyl groups for B, mention may be made of:

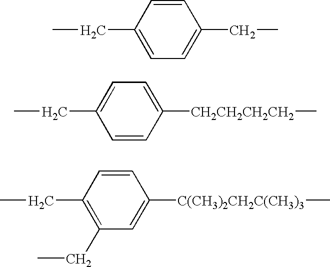

As monoxoaralkyl groups for B, mention may be made of:

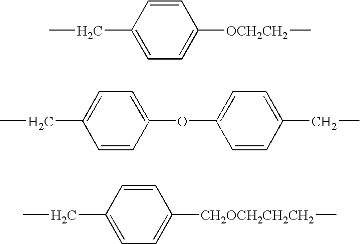

The preferred compounds of formula (I) are chosen from those for which:

R¹ and R², which may be identical or different, denote hydrogen, a C₁-C₈ alkyl radical, a C₁-C₈ alkoxy radical or a water-solubilizing substituent chosen from a carboxylate group, a sulphonate group and an ammonium residue;

R³ and R⁴, which may be identical or different, denote a group COOR⁶ or CN;

R⁵ denotes a C₁-C₆ alkyl radical;

R⁶ denotes a C₁-C₁₂ alkyl radical;

A denotes O;

B denotes a C₁-C₁₂ alkylene radical, a C₄-C₁₂ cycloalkylene radical, a C₈-C₂₂ aralkylene radical or a group [X]ₙ—Y—;

X denotes a group —CH₂—CH₂—)—, —CH₂CH₂CH₂O—, —CH(CH₃)—CH₂—O—, —CH₂—CH₂—CH₂—CH₂—O— or CH₂—CH(CH₂CH₃)—O—;

Y denotes a group —CH₂—CH₂—, —CH₂CH₂CH₂—, —CH(CH₃)—CH₂—, —CH₂—CH₂—CH₂—CH₂— or —CH₂—CH(CH₂CH₃)—;

n ranges from 1 to 20.

The compounds of formula (I) that are more preferred are chosen from those for which:

R¹ and R², which may be identical or different, denote hydrogen, a C₄-C₈ alkyl radical, a C₄-C₈ alkoxy radical or a water-solubilizing substituent chosen from a carboxylate group, a sulphonate group and an ammonium residue;

R³ and R⁴ denote a group CN;

R⁵ denotes a C₁-C₆ alkyl radical;

A denotes O;

B denotes a C₁-C₁₂ alkylene radical or a group [X]ₙ—Y—;

R⁸ denotes hydrogen or a C₁-C₁₂ alkyl radical;

X denotes a group —CH₂—CH₂—O— or —CH(CH₃)—CH₂—O—,

Y denotes a group —CH₂—CH₂— or —CH(CH₃)—CH₂—;

n ranges from 1 to 20.

The compounds of formula (I) that are even more particularly preferred are chosen from those corresponding to formula (Ia) below:

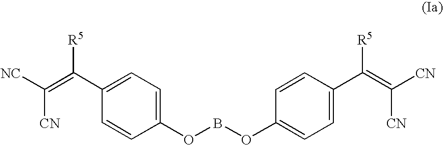

in which R⁵ is a C₁-C₆ alkyl radical and more particularly methyl; B denotes a C₁-C₁₂ alkylene radical.

As examples of compounds of formula (I) in accordance with the invention, mention may be made of those having the formula below:

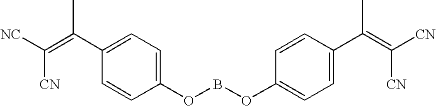

in which B is chosen from the following groups:

—(CH₂)₂—

—(CH₂)₃—

—(CH₂)₄—

—(CH₂)₅—

—(CH₂)₆—

—(CH₂)₇—

—(CH₂)₈—

—(CH₂)₉—

—(CH₂)₁₀—

—(CH₂)₁₂—

—CH₂—CH₂—CH—(CH₃)—CH₂—CH₂—

—CH₂—C(CH₃)₂—CH₂—

—CH₂—CH(OH)—CH₂—

—CH₂—CH(OH)—CH(OH)—CH₂—

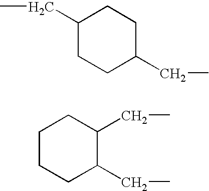

Among these compounds, the two compounds below will be chosen more particularly:

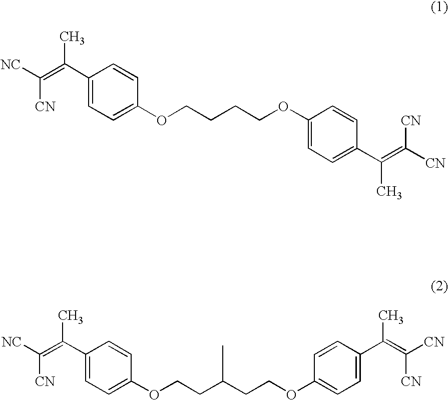

The compounds of formula (I) as defined above are known per se and their structures and syntheses are described in patent application DE 198 55 649 (which forms an integral part of the content of the description).

The compounds of formula (I) are generally present in the composition of the invention in proportions of between 0.1% and 20% by weight and preferably between 0.5% and 10% by weight relative to the total weight of the composition.

As mentioned above, the dibenzoylmethane derivatives intended to be photo-stabilized in the context of the present invention are products that are already well known per se and described in particular in the abovementioned documents FR 2 326 405, FR 2 440 933 and EP 0 114 607, the teachings of which documents are, as regards the actual definition of these products, entirely included as references in the present description.

According to the present invention, one or more dibenzoylmethane derivatives may obviously be used.

Among the dibenzoylmethane derivatives which fall particularly suitably into the context of the present invention, mention may be made in particular, in a non-limiting manner, of:

2-methyldibenzoylmethane

4-methyldibenzoylmethane

4-isopropyldibenzoylmethane

4-tert-butyldibenzoylmethane

2,4-dimethyldibenzoylmethane

2,5-dimethyldibenzoylmethane

4,4′-diisopropyldibenzoylmethane

4,4′-dimethoxydibenzoylmethane

4-tert-butyl-4′-methoxydibenzoylmethane

2-methyl-5-isopropyl-4′-methoxydibenzoylmethane

2-methyl-5-tert-butyl-4′-methoxydibenzoylmethane

2,4-dimethyl-4′-methoxydibenzoylmethane

2,6-dimethyl-4-tert-butyl-4′-methoxydibenzoylmethane.

Among the dibenzoylmethane derivatives mentioned above, it is most particularly preferred to use, according to the present invention, 4-(tert-butyl)-4′-methoxydibenzoylmethane, in particular the product sold under the trade name “Parsol 1789” by the company Givaudan, this screening agent thus corresponding to the following structural formula:

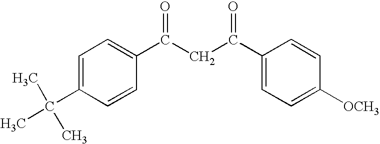

Another dibenzoylmethane derivative which is preferred according to the present invention is 4-isopropyldibenzoylmethane, this screening agent being sold under the name “Eusolex 8020” by the company Merck and corresponding to the following structural formula:

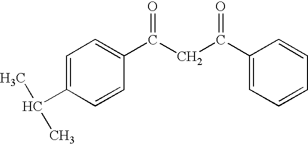

The dibenzoylmethane derivative(s) may be present in the compositions in accordance with the invention, or in the compositions intended to be stabilized in accordance with the process of the invention, in contents that are generally between 0.1% and 20% by weight and preferably in contents of between 0.5% and 10% by weight relative to the total weight of the composition.

The compositions in accordance with the invention may additionally comprise other additional UVA-active and/or UVB-active organic UV sunscreens (absorbers) which are water-soluble or liposoluble or alternatively insoluble in the cosmetic solvents commonly used.

The additional organic UV sunscreens are chosen in particular from anthranilates; salicylic derivatives; camphor derivatives; benzophenone derivatives; triazine derivatives such as those described in patent applications U.S. Pat. Nos. 4,367,390, 4,724,137, EP 863 145, EP 517 104, EP 570 838, EP 796 851, EP 775 698, EP 878 469 and EP 933 376, EP 507 691, EP-507 692, EP 790 243 and EP 944 624; β,β′-diphenylacrylate derivatives; benzotriazole derivatives; benzalmalonate derivatives; benzimidazole derivatives; imidazolines; bis-benzazolyl derivatives as described in patents EP 669 323 and U.S. Pat. No. 2,463,264; p-aminobenzoic acid derivatives (PABA); methylene bis(hydroxyphenyl benzotriazole) derivatives as described in patent applications U.S. Pat. Nos. 5,237,071, 5,166,355, GB 2 303 549, DE 197 26 184 and EP 893 119; screening polymers and screening silicones such as those described in particular in patent application WO 93/04665; 4,4-diarylbutadienes such as those described in patent applications EP 0 967 200 and DE 197 55 649; and mixtures thereof.

As examples of additional UV-A-active and/or UV-B-active organic sunscreens, mention may be made of those designated below under their INCI name:

para-Aminobenzoic Acid Derivatives:

PABA,

Ethyl PABA,

Ethyl dihydroxypropyl PABA,

Ethylhexyl dimethyl PABA sold in particular under the name “Escalol 507” by ISP,

Glyceryl PABA,

PEG-25 PABA sold under the name “Uvinul P25” by BASF,

Salicylic Derivatives:

Homosalate sold under the name “Eusolex HMS” by RONA/EM Industries,

Ethylhexyl salicylate sold under the name “Neo Heliopan OS” by Haarmann and Reimer,

Dipropylene glycol salicylate sold under the name of “Dipsal” by Scher,

TEA salicylate sold under the name “Neo Heliopan TS” by Haarmann and Reimer,

β,β′-Diphenylacrylate Derivatives:

Octocrylene sold in particular under the trade name “Uvinul N539” by BASF,

Etocrylene, sold in particular under the trade name “Uvinul N35” by BASF,

Benzophenone Derivatives:

Benzophenone-1 sold under the trade name “Uvinul 400” by BASF,

Benzophenone-2 sold under the trade name “Uvinul D50” by BASF,

Benzophenone-3 or Oxybenzone, sold under the trade name “Uvinul M40” by BASF,

Benzophenone-4 sold under the trade name “Uvinul MS40” by BASF,

Benzophenone-5

Benzophenone-6 sold under the trade name “Helisorb 11” by Norquay

Benzophenone-8 sold under the trade name “Spectra-Sorb UV-24” by American Cyanamid,

Benzophenone-9 sold under the trade name “Uvinul DS-49” by BASF,

Benzophenone-12

Benzylidenecamphor Derivatives:

3-Benzylidenecamphor manufactured under the name “Mexoryl SD” by Chimex,

Benzylidenecamphorsulphonic acid manufactured under the name “Mexoryl SL” by Chimex,

Camphorbenzalkonium methosulphate manufactured under the name “Mexoryl SO” by Chimex,

Terephthalylidenedicamphorsulphonic acid manufactured under the name “Mexoryl SX” by Chimex,

Polyacrylamidomethylbenzylidenecamphor manufactured under the name “Mexoryl SW” by Chimex,

Benzimidazole Derivatives:

Phenylbenzimidazolesulphonic acid sold in particular under the trade name “Eusolex 232” by Merck,

Disodium phenyldibenzimidazoletetrasulphonate sold under the trade name “Neo Heliopan AP” by Haarmann and Reimer,

Benzotriazole Derivatives:

Methylene bis(benzotriazolyl)tetramethylbutylphenol, sold in solid form under the trade name “Mixxim BB/100” by Fairmount Chemical, or in micronized form as an aqueous dispersion under the trade name “Tinosorb M” by Ciba Specialty Chemicals,

Drometrizole trisiloxane sold under the name “Silatrizole” by Rhodia Chimie,

Triazine Derivatives:

Anisotriazine sold under the trade name “Tinosorb S” by Ciba Specialty Chemicals

Ethylhexyltriazone sold in particular under the trade name “Uvinul T150” by BASF,

Diethylhexylbutamidotriazone sold under the trade name “Uvasorb HEB” by Sigma 3V,

2,4,6-tris(diisobutyl 4′-aminobenzalmalonate)-s-triazine

Anthranilic Derivatives:

Menthyl anthranilate sold under the trade name “Neo Heliopan MA” by Haarmann and Reimer,

Imidazoline Derivatives:

Ethylhexyl dimethoxybenzylidenedioxoimidazoline-propionate,

Benzalmalonate Derivatives:

Polyorganosiloxane containing a benzalmalonate function, sold under the trade name “Parsol SLX” by Hoffmann La Roche, and mixtures thereof.

The additional organic UV screening agents that are more particularly preferred are chosen from the following compounds:

Ethylhexyl salicylate,

Octocrylene,

Phenylbenzimidazolesulphonic acid,

Terephthalylidenedicamphorsulphonic acid,

Anisotriazine,

Ethylhexyltriazone,

Diethylhexylbutamidotriazone,

2,4,6-Tris(diisobutyl 4′-aminobenzalmalonate)-s-triazine

Benzophenone-3,

Benzophenone-4,

Benzophenone-5,

Disodium phenyldibenzimidazoletetrasulphonate,

Methylenebis(benzotriazolyl)tetramethylbutylphenol,

Drometrizole

and mixtures thereof.

The cosmetic compositions according to the invention may also contain pigments or nanopigments (average size of the primary particles: generally between 5 nm and 100 nm and preferably between 10 nm and 50 nm) of coated or uncoated metal oxides, such as, for example, nanopigments of titanium oxide (amorphous or crystallized in rutile and/or anatase form), of iron oxide, of zinc oxide, of zirconium oxide or of cerium oxide, which are all UV photo-protective agents that are well known per se. Standard coating agents are, moreover, alumina and/or aluminium stearate. Such coated or uncoated metal oxide nanopigments are described in particular in patent applications EP-A-0 518 772 and EP-A-0 518 773.

The compositions according to the invention may also contain agents for artificially tanning and/or browning the skin (self-tanning agents) such as, for example, dihydroxyacetone (DHA).

The compositions of the invention may also comprise standard cosmetic adjuvants chosen in particular from fatty substances, organic solvents, ionic or nonionic thickeners, softeners, antioxidants, free-radical scavengers, opacifiers, stabilizers, emollients, silicones, α-hydroxy acids, antifoams, moisturizers, vitamins, insect repellents, fragrances, preserving agents, surfactants, antiinflammatories, substance P antagonists, fillers, polymers, propellants, acidifying or basifying agents and colorants, or any other ingredient usually used in cosmetics, in particular for manufacturing antisun compositions in the form of emulsions.

The fatty substances may consist of an oil or a wax or mixtures thereof, and they also comprise fatty acids, fatty alcohols and linear or cyclic fatty acid esters such as benzoic, trimellitic and hydroxybenzoic acid derivatives. The oils may be chosen from animal, plant, mineral and synthetic oils and in particular from liquid petroleum jelly, liquid paraffin, volatile or non-volatile silicone oils, isoparaffins, polyolefins, fluoro oils and perfluoro oils. Similarly, the waxes may be chosen from animal, fossil, plant, mineral and synthetic waxes that are known per se.

Among the organic solvents which may be mentioned are lower alcohols and polyols.

Needless to say, a person skilled in the art will take care to select this or these optionally additional compound(s) and/or the amounts thereof such that the advantageous properties, in particular the photostability, intrinsically associated with the compositions in accordance with the invention are not, or are not substantially, adversely affected by the addition(s) envisaged.

The compositions of the invention may be prepared according to techniques that are well known to those skilled in the art, in particular those intended for preparing emulsions of oil-in-water or water-in-oil type.

These compositions may be in particular in the form of a simple or complex (O/W, W/O, O/W/O or W/O/W) emulsion-such as a cream, a milk, a gel, a cream-gel, a powder or a solid tube, and may optionally be packaged as an aerosol and may be in the form of a mousse or a spray.

When it is an emulsion, the aqueous phase thereof may comprise a nonionic vesicular dispersion prepared according to known processes (Bangham, Standish and Watkins. J. Mol. Biol. 13, 238 (1965), FR 2 315 991 and FR 2 416 008).

The cosmetic composition of the invention may be used as a composition for protecting the human epidermis or the hair against ultraviolet rays, as an antisun composition or as a make-up product.

When the cosmetic composition according to the invention is used for protecting the human epidermis against UV rays, or as an antisun composition, it may be in the form of a suspension or a dispersion in solvents or fatty substances, in the form of a nonionic vesicular dispersion or in the form of an emulsion, preferably of oil-in-water type, such as a cream or a milk, or in the form of an ointment, a gel, a cream-gel, a solid tube, a powder, a stick, an aerosol mousse or a spray.

When the cosmetic composition according to the invention is used for protecting the hair against UV rays, it may be in the form of a shampoo, a lotion, a gel, an emulsion or a nonionic vesicular dispersion and may constitute, for example, a rinse-out composition, to be applied before or after shampooing, before or after dyeing or bleaching or before, during or after permanent-waving or relaxing the hair, a styling or treating lotion or gel, a blow-drying or hairsetting lotion or gel, or a permanent-waving, relaxing, dyeing or bleaching composition for the hair.

When the composition is used as a make-up product for the eyelashes, eyebrows or the skin, such as an epidermal treatment cream, a foundation, a tube of lipstick, an eyeshadow, a-face powder, a mascara or an eyeliner, it may be in solid or pasty, anhydrous or aqueous form, for instance oil-in-water or water-in-oil emulsions, nonionic vesicular dispersions or suspensions.

As a guide, for the antisun formulations in accordance with the invention which have a support of oil-in-water emulsion type, the aqueous phase (in particular comprising the hydrophilic screening agents) generally represents from 50% to 95% by weight and preferably from 70% to 90% by weight, relative to the total weight of the formulation, the oily phase (in particular comprising the lipophilic screening agents) preferably represents from 5% to 50% by weight and preferably from 10% to 30% by weight, relative to the total weight of the formulation, and the (co)emulsifier(s) generally represent from 0.5% to 20% by weight and preferably from 2% to 10% by weight relative to the total weight of the formulation.

As mentioned at the start of the description, one subject of the invention is the use of a composition as defined above for manufacturing a cosmetic or dermatological composition intended for the protection of skin and/or hair against ultraviolet radiation, in particular solar radiation.

Another subject of the present invention lies in a process for improving the stability of at least one dibenzoylmethane derivative towards UV radiation which consists in combining with the said dibenzoylmethane derivative an effective amount of at least one UV screening agent of the α-alkylstyrene-based dimer type of formula (I) as defined above.

Another subject of the present invention consists of the use of a UV screening agent of α-alkylstyrene-based dimer type of formula (I) as defined above, in the preparation of a cosmetic or dermatological composition comprising at least one UV screening agent of the dibenzoylmethane-based type, with the aim of improving the stability towards UV rays of the said dibenzoylmethane derivative.

Concrete examples, which illustrate the invention without implying any limitation whatsoever, will now be given.

### EXAMPLES

| O/W emulsion COMPOSITION | Ex. 1 | |
|---|---|---|
| Glyceryl mono/distearate/polyethylene glycol | 2 | g |
| stearate (100 EO) mixture (Arlacel 165 FL- | | |
| ICI) | | |
| Stearyl alcohol | 1 | g |
| (Lanette 18-Henkel) | | |
| Stearic acid from palm oil | 1.5 | g |
| (Stearine TP-Stearinerie Dubois) | | |
| Polydimethylsiloxane | 0.5 | g |
| (Dow Corning 200 Fluid-Dow Corning) | | |
| C12/C15 alkylbenzoate | 5 | g |
| (Wtconol TN-WITCO) | | |
| Oxyethylenated oxypropylenated | 1 | g |
| polydimethyl/methylsiloxane as a 10% solution | | |
| in D5 | | |
| (DC 5225 C-Dow Corning) | | |
| Polymethylphenyl | 3 | g |
| (Mirasil PTM-Rhodia Chemie) | | |
| Triethanolamine | 0.5 | g |
| Butylmethoxydibenzoylmethane | 2 | g |
| (Parsol 1789-Givaudan) | | |
| Compound of formula (1) as defined above | 8 | g |
| Titanium oxide | 2.5 | g |
| (Titanium dioxide MT-100 TV Tayca) | | |
| Glycerol | 4 | g |
| Hexadecyl phosphate, potassium salt | 1 | g |
| (Amphisol K-Hoffman LaRoche) | | |
| Polyacrylic acid | 0.3 | g |
| (Synthalen K-3V) | | |
| Hydroxypropylmethylcellulose | 0.1 | g |
| (Methocel F4M-Dow Chemical) | | |
| Triethanolamine | pH: 7 | qs |
| Preserving agents | | qs |
| Demineralized waterqs | 100 | g |
| W/O COMPOSITION | Ex. 2 | |
| Oxyethylenated polydimethyl/methylcetyl- | 2 | g |
| methylsiloxane | | |
| (Abil EM 90D-Goldschmidt) | | |
| Phenyltrimethylsiloxytrisilxane | 3 | g |
| (Dow Corning 556 Cosmetic Grade Fluid-Dow | | |
| Corning) | | |
| C₁₂/C₁₅ alkylbenzoate | 8 | g |
| (Witconol TN-Witco) | | |
| Drometrizole trisiloxane | 2 | g |
| (Silatrizole-Rhodia Chimie) | | |
| Butylmethoxydibenzoylmethane | 2 | g |
| (Parsol 1789-Glivaudan) | | |
| Compound of formula (2) as defined above | 6 | g |
| Titanium oxide | 3 | g |
| (Titanium dioxide MT 100 TV-Tayca) | | |
| Glycerol | 5 | g |
| Magnesium sulphate | 0.7 | g |
| Preserving agents | | qs |
| Demineralized waterqs | 100 | g |
| O/W COMPOSITION | Ex. 3 | |
| 80/20 mixture of cetylstearyl alcohol and of | 7 g | |
| oxyethylenated (33 EO) cetylstearyl alcohol | | |
| (Sinnowzx AO-Henkel) | | |
| Mixture of glyceryl mono- and distearate | 2 g | |
| (Cerasynt SD-V ISP) | | |
| Cetyl alcohol | 1.5 g | |
| Polydimethylsiloxane | 1 g | |
| (Dow Corning 200 fluid-Dow Corning) | | |
| C₁₂/C₁₅ alkylbenzoate | 15 g | |
| (Witconol TN-Witco) | | |
| Bisethylhexyloxyphenol methoxyphenyltriazine | 2 g | |
| (Tinosorb S-Ciba) | | |
| Glycerol | 15 g | |
| Disodium phenyldibenzimidazoletetrasulphonate | 1.5 g | |
| (Neoheliopan AP-Haarmann and Reimer) | | |
| Terephthalylidenedicamphorsulphonic acid | 2 g | |
| (Mexoryl SX-Chimex) | | |
| Butylmethoxydibenzoylmethane | 2 g | |
| (Parsol 1789-Givaudan) | | |
| Compound of formula (2) as defined above | 6 g | |
| Triethanolamine | qs | pH 7 |
| Preserving agents | qs | |
| Demineralized waterqs | 100 g | |

1. Cosmetic or dermatological composition, for topical use, characterized in that it comprises at least, in a cosmetically acceptable support:
(a) from 0.1 to 20% by weight of a UV screening agent of the dibenzoylmethane-based type and
(b) from 0.1 to 20% by weight of an α-alkylstyrene-based dimer of formula (I) below: 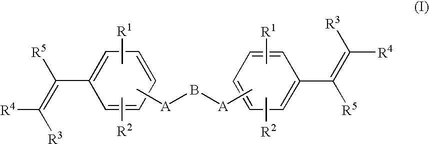
in which
R¹ and R², which may be identical or different, denote hydrogen, OH, NH₂, a linear or branched C₁-C₁₂ alkyl radical; a linear or branched C₁-C₁₂ alkoxy radical; a linear or branched C₁-C₁₂ monoalkylamino radical; a linear or branched C₁-C₁₂ dialkylamino radical or a water-solubilizing substituent chosen from a carboxylate group, a sulphonate group and an ammonium residue;
R³ and R⁴, which may be identical or different, denote a group COOR⁶, COR⁶, CONR⁶R⁷ or CN;
R⁵ denotes a linear or branched C₁-C₁₂ alkyl radical;
R⁶ and R⁷, which may be identical or different, denote hydrogen, a linear or branched C₁-C₁₂ alkyl radical; a linear or branched C₂-C₁₀ alkenyl radical; a branched or unbranched C₃-C₁₀ cycloalkyl radical; a branched or unbranched C₃-C₁₀ cycloalkenyl radical; a C₆-C₁₈ aryl or a C₄-C₇ heteroaryl; the said cycloalkyl, cycloalkenyl and aryl groups possibly comprising one or more substituents chosen from halogens, a cyano group, a nitro group, an amino group, a C₁-C₄ monoalkylamino radical, a C₁-C₄ dialkylamino radical, a hydroxyl group, a C₁-C₄ alkyl radical and a C₁-C₄ alkoxy radical; the said cycloalkyl and cycloalkenyl groups possibly comprising one or more hetero atoms (for example O, N or S);
A denotes O, S or a group NR⁸;
R⁸ denotes hydrogen or a linear or branched C₁-C₁₂ alkyl radical;
B denotes a linear or branched C₁-C₁₂ alkylene radical which may comprise one or more substituents chosen from hydroxyl, OC₁-C₆-acyl, NH₂, NH-C₁-C₆-alkyl, NH-C₁-C₆-acyl, CN, COOH and COO-C₁-C₆-acyl; a branched or unbranched C₄-C₁₂ cycloalkylene radical; a C₈-C₂₂ aralkylene radical; a C₉-C₂₁ monooxoaralkylene radical; or a group [X]ₙ—Y—;
at least two of the radicals R¹, R² and R⁸ may together form a 5- or 6-membered ring with the benzene nucleus to which they are attached;
X denotes a group —CH₂—CH₂—Z—, —CH₂CH₂CH₂Z—, —CH(CH₃)—CH₂—Z—, —CH₂—CH₂—CH₂—CH₂—Z— or CH₂—CH(CH₂CH₃)—Z—;
Y denotes a group —CH₂—CH₂—, —CH₂CH₂CH₂—, —CH(CH₃)—CH₂—, —CH₂—CH₂—CH₂—CH₂— or —CH₂—CH(CH₂CH₃)—;
Z denotes O or S; n ranges from 1 to 150.

2. Composition according to claim 1, in which the compounds of formula (I) are chosen from those for which:
R¹ and R², which may be identical or different, denote hydrogen, a C₁-C₈ alkyl radical, a C₁-C₈ alkoxy radical or a water-solubilizing substituent chosen from a carboxylate group, a sulphonate group and an ammonium residue;
R³ and R⁴, which may be identical or different, denote a group COOR⁶ or CN;
R⁵ denotes a C₁-C₆ alkyl radical;
R⁶ denotes a C₁-C₁₂ alkyl radical;
A denotes O;
B denotes a C₁-C₁₂ alkylene radical, a C₄-C₁₂ cycloalkylene radical, a C₈-C₂₂ aralkylene radical or a group [X]ₙ—Y—;
X denotes a group —CH₂—CH₂—O—, —CH₂CH₂CH₂O—, —CH(CH₃)—CH₂—O—, —CH₂—CH₂—CH₂—CH₂—O— or CH₂—CH(CH₂CH₃)—O—;
Y denotes a group —CH₂—CH₂—, —CH₂CH₂CH₂—, —CH(CH₃)—CH₂—, —CH₂—CH₂—CH₂—CH₂— or —CH₂—CH(CH₂CH₃)—;
n ranges from 1 to 20.

3. Composition according to claim 1 or 2, in which the compounds of formula (I) are chosen from those for which:
R¹ and R², which may be identical or different, denote hydrogen, a C₄-C₈ alkyl radical, a C₄-C₈ alkoxy radical or a water-solubilizing substituent chosen [lacuna] a carboxylate group, a sulphonate group and an ammonium residue;
R³ and R⁴ denote a CN group;
R⁵ denotes a C₁-C₆ alkyl radical;
A denotes O;
B denotes a C₁-C₁₂ alkylene radical or a group [X]ₙ—Y—;
R⁸ denotes hydrogen or a C₁-C₁₂ alkyl radical;
X denotes a group —CH₂—CH₂—O— or —CH(CH₃)—CH₂—O—;
Y denotes a group —CH₂—CH₂— or —CH(CH₃)—CH₂
n ranges from 1 to 20.

4. Composition according to any one of claims 1 to 3, in which the compounds of formula (I) are chosen from those corresponding to formula (Ia) below: 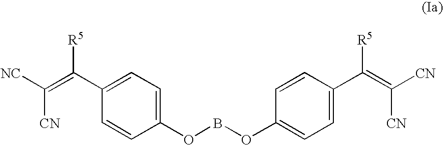
in which R⁵ is a C₁-C₆ alkyl radical and more particularly methyl; B denotes a C₁-C₁₂ alkylene radical.

5. Composition according to claim 4, in which the compounds of formula (I) are chosen from those of the following formula: 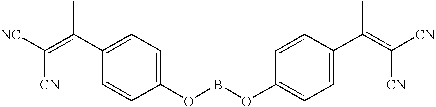
in which B is chosen from the following groups:
—(CH₂)₂—
—(CH₂)₃—
—(CH₂)₄—
—(CH₂)₅—
—(CH₂)₆—
—(CH₂)₇—
—(CH₂)₈—
—(CH₂)₉—
—(CH₂)₁₀—
—(CH₂)₁₂—
—CH₂—CH₂—CH—(CH₃)—CH₂—CH₂—
—CH₂—C(CH₃)₂—CH₂—
—CH₂—CH(OH)—CH₂—
—CH₂—CH(OH)—CH(OH)—CH₂—

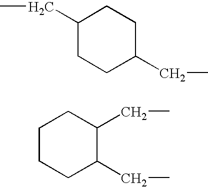

6. Composition according to claim 5, in which the compounds of formula (I) are chosen from the following compounds:

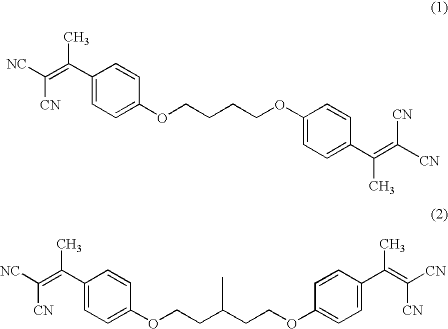

7. Composition according to any one of claims 1 to 6, characterized in that the α-alkylstyrene-based dimer is present in concentrations ranging from 0.5 to 10% by weight relative to the total weight of the composition.

8. Composition according to any one of claims 1 to 7, characterized in that the dibenzoylmethane derivative is present in concentrations ranging from 0.5 to 10% by weight relative to the total weight of the composition.

9. Composition according to any one of claims 1 to 8, characterized in that the dibenzoylmethane derivative is chosen from:
2-methyldibenzoylmethane
4-methyldibenzoylmethane
4-isopropyldibenzoylmethane
4-tert-butyldibenzoylmethane
2,4-dimethyldibenzoylmethane
2,5-dimethyldibenzoylmethane
4,4′-diisopropyldibenzoylmethane
4,4′-dimethoxydibenzoylmethane
4-tert-butyl-4′-methoxydibenzoylmethane
2-methyl-5-isopropyl-4′-methoxydibenzoylmethane
2-methyl-5-tert-butyl-4′-methoxydibenzoylmethane
2,4-dimethyl-4′-methoxydibenzoylmethane
2,6-dimethyl-4-tert-butyl-4′-methoxydibenzoylmethane.

10. Composition according to claim 9, characterized in that the dibenzoylmethane derivative is 4-(tert-butyl)-4′-methoxydibenzoylmethane.

11. Composition according to claim 9, characterized in that the dibenzoylmethane derivative is 4-isopropyldibenzoylmethane.

12. Composition according to any one of claims 1 to 11, characterized in that it also contains other UV-A-active and/or UV-B-active organic screening agents.

13. Composition according to claim 12, in which the additional organic UV screening agent(s) is (are) chosen from anthranilates; salicylic derivatives, camphor derivatives; benzophenone derivatives; triazine derivatives; β,β′-diphenylacrylate derivatives; benzotriazole derivatives; benzalmalonate derivatives; benzimidazole derivatives; imidazolines; bis-benzoazolyl derivatives; p-aminobenzoic acid (PABA) derivatives; methylene bis(hydroxyphenylbenzotriazole) derivatives; screening polymers and screening silicones; and 4,4-diarylbutadienes, and mixtures thereof.

14. Composition according to claim 13, characterized in that the organic UV screening agent(s) is (are) chosen from the following compounds:
Ethylhexyl salicylate,
Octocrylene,
Phenylbenzimidazolesulphonic acid,
Terephthalylidenedicamphorsulphonic acid,
Anisotriazine,
Ethylhexyltriazone,
Diethylhexylbutamidotriazone,
2,4,6-Tris(diisobutyl 4′-aminobenzalmalonate)-s-triazine
Benzophenone-3,
Benzophenone-4,
Benzophenone-5,
Disodium phenyldibenzimidazoletetrasulphonate,
Methylenebis(benzotriazolyl)tetramethylbutylphenol,
Drometrizole
and mixtures thereof.

15. Composition according to any one of claims 1 to 14, characterized in that it also comprises coated or uncoated metal oxide pigments or nanopigments.

16. Composition according to claim 15, characterized in that the said pigments or nanopigments are chosen from titanium oxide, zinc oxide, iron oxide, zirconium oxide and cerium oxide, and mixtures thereof, that may be coated or uncoated.

17. Composition according to any one of claims 1 to 16, characterized in that it also comprises at least one agent for artificially tanning and/or bronzing the skin.

18. Composition according to any one of claims 1 to 17, characterized in that it also comprises at least one adjuvant chosen from fatty substances, organic solvents, ionic or nonionic thickeners, softeners, antioxidants, free-radical scavengers, opacifiers, stabilizers, emollients, silicones, α-hydroxy acids, antifoams, moisturizers, vitamins, insect repellents, fragrances, preserving agents, surfactants, anti-inflammatories, substance P antagonists, fillers, polymers, propellants, acidifying or basifying agents, and colorants.

19. Composition according to any one of claims 1 to 18, characterized in that it is a protective composition for the human epidermis or an antisun composition and in that it is in the form of a nonionic vesicular dispersion an emulsion, in particular an emulsion of oil-in-water type, a cream, a milk, a gel, a cream-gel, a suspension, a dispersion, a powder, a solid tube, a mousse or a spray.

20. Composition according to any one of claims 1 to 18, characterized in that it is a make-up composition for the eyelashes, the eyebrows or the skin and in that it is in solid or pasty, anhydrous or aqueous form, or in the form of an emulsion, a suspension or a dispersion.

21. Composition according to any one of claims 1 to 18, characterized in that it is a composition intended for protecting the hair against ultraviolet rays and in that it is in the form of a shampoo, a lotion, a gel, an emulsion or a nonionic vesicular dispersion.

22. Use of a composition as defined in claims 1 to 18, for the manufacture of cosmetic or dermatological compositions for protecting the skin and/or the hair against ultraviolet radiation, in particular solar radiation.

23. Process for improving the stability of at least one dibenzoylmethane derivative towards UV radiation, characterized in that it consists in combining with the said dibenzoylmethane derivative an effective amount of at least one dimer derived from α-alkylstyrene of formula (I) as defined according to any one of claims 1 to 6.

24. Use of an α-alkylstyrene-based dimer of formula (I) as defined according to any one of claims 1 to 6, in the preparation of a cosmetic or dermatological composition containing at least one dibenzoylmethane derivative, to improve the stability of the said dibenzoylmethane derivative towards UV radiation.
